(19) United States
(12) Patent Application Publication
(10) Pub. No.: US 2014/0356338 A1
(43) Pub. Date: Dec. 4, 2014

(54) RICE-FERMENTED FOOD COMPOSITION CONTAINING RICE SACCHARIFIED LIQUID FERMENTED WITH KIMCHI LACTIC ACID BACTERIA AS ACTIVE INGREDIENT AND HAVING ANTIBACTERIAL AND ANTIVIRAL EFFECTS

(71) Applicant: PROBIONIC Co., Ltd., Deajeon (KR)

(72) Inventors: Yong-Ha Park, Daejeon (KR); Jeong-Heui Lim, Daejeon (KR); Byoung-Joo Seo, Daegu (KR)

(21) Appl. No.: 14/115,458

(22) PCT Filed: May 12, 2011
(86) PCT No.: PCT/KR2011/003522

§ 371 Date: Jan. 14, 2014

(30) Foreign Application Priority Data
May 12, 2011 (KR) 10-2011-0044557

(51) Int. Cl. A61K 35/74; A23L 1/105

(52) U.S. Cl. CPC A61K 35/747; A23L 1/105; A23Y 2220/77; A61K 2035/11
USPC 424/93.45; 435/252.9

(57) ABSTRACT

Disclosed are a rice-fermented food composition which contains a rice saccharified liquid fermented with kimchi lactic acid bacteria as an active ingredient and has antibacterial and antiviral effects. More particularly, the present invention relates to a rice lactic acid bacteria fermented food composition which is prepared by adding kimchi lactic acid bacteria to a rice saccharified liquid as a main ingredient and fermenting the mixture, and has antibacterial and antiviral effects against atopic dermatitis and avian influenza virus, and a functional health food comprising the same.

### REFERENCE TO RELATED APPLICATIONS

This application is a U.S. National Phase application of International Application No. PCT/KR2011/003522, filed May 12, 2011, and claims priority to Korean Patent Application No. 10-2011-0044557, filed May 12, 2011, the disclosures of each of these applications being incorporated herein by reference in their entireties.

### FIELD OF THE INVENTION

The present invention relates to a rice-fermented food composition which contains a rice saccharified liquid fermented with kimchi lactic acid bacteria as an active ingredient and has antibacterial and antiviral effects. More particularly, the present invention relates to a rice lactic acid bacteria fermented food composition which is prepared by adding kimchi lactic acid bacteria to a rice saccharified liquid as a main ingredient and fermenting the mixture, and has antibacterial and antiviral effects against atopic dermatitis and avian influenza virus, and a functional health food comprising the same.

### BACKGROUND OF THE INVENTION

Lactic acid bacteria had been used for foods in Korea for a long time and the most representative food using lactic acid bacteria is kimchi. As excellence of kimchi which is a traditional Korean fermented food was known to the whole world and Codex standard of kimchi was firstly adopted as a Korean food in the 24th general meeting of Codex Alimentarius Commission on Jul. 5, 2001.

It was found that lactic acid bacteria constituting kimchi are three genera of Lactobacillus, Leuconostoc and Weissella, and representative lactic acid bacteria species are Lactobacillus sakei subsp. sakei, Leuconostoc citreum and Weissella koreensis.

The composition of these species may be changed according to internal and external environments of kimchi and types of lactic acid bacteria may vary according to kimchi. Accordingly, the taste of kimchi may also greatly depend on fermentation by-products obtained from metabolism of kimchi lactic acid bacteria. As fermentation proceeds, harmful aerobic bacteria present in kimchi are killed, anaerobic beneficial bacteria propagate, lactic acid concentration increases, acidity is reduced and more lactic acids are gradually proliferated. These kimchi lactic acid bacteria strengthen in vivo immune function and have an antibacterial activity of inhibiting harmful bacteria through production of bacteriocins, organic acids and the like, thereby contributing to production and maintenance of normal intestinal microflora. Furthermore, kimchi lactic acid bacteria are also known to have blood cholesterol reduction and anti-cancer effects.

Among lactic acid bacteria isolated from kimchi, Lactobacillus sakei belongs to a lactic acid bacteria (LAB) group which produces organic acids. In general, lactobacillus is found predominantly in meat as well as in plant fermentation products and fermented fish. This bacterium is a commercially essential bacterium, which is used as a starter for making fermented sausage products and as a biopreservative culture of meat and fish foods. Lactobacillus sakei produces organic acids as primary metabolites, has an important action as a starter and a preservative culture, preserves foods based on acidification of organic acids and contributes to improvement of product quality. In addition, Lactobacillus sakei has antibacterial activity through production of bacteriocins. This antibacterial activity is an antibacterial activity against fish pathogenic bacteria, which aids in prevention of bacterial diseases of fish and inhibits listeria during meat fermentation.

Accordingly, the present inventors succeeded in finding a strain of Lactobacillus sakei which has potent allergy prevention efficacy, among Lactobacillus sakei which is a lactic acid bacterium rich in kimchi and generally used for fermentation of sausages and meat. The present inventors succeeded in isolating, from kimchi, Lactobacillus sakei ProBio-65 with acid resistance, biliary resistance, antibacterial activity and antibiotic resistance, as a subspecies of Lactobacillus sakei.

That is, the developed strain was obtained by diluting kimchi juice stepwise, culturing the kimchi juice in lactic acid bacteria selection medium, isolating only lactic acid bacteria from the medium, selecting the isolated bacteria, culturing the bacteria in a MRS medium and finally selecting a microbe potently inhibiting proliferation of Staphylococcus aureus which is an aggravating factor in atopic dermatitis, which is called “Lactobacillus sakei ProBio-65” (accession number: KCTC 10755BP) and was patented as Korean Patent No. 0479719.

The kimchi-derived lactic acid bacteria, Lactobacillus sakei ProBio-65 has a superior function of suppressing growth of staphylococcus aureus known as a disease-aggravating factor of atopic dermatitis as well as antibacterial activities against various pathogenic bacteria, superior inhibitory activity of IgE production and excellent activation capability of immune systems. In addition, Lactobacillus sakei efficiently reaches intestines and contributes to growth suppression of harmful bacteria and normalization of intestinal microflora through secretion of antibacterial substances due to superior acid resistance and bile acid resistance.

Meanwhile, atopic dermatitis is an inflammatory skin disease, which causes serious pruritus and pathognomonic eczematous skin lesions, and is predominantly onset in infancy and childhood. Atopic dermatitis is an atopic disease which is chronically onset, in particular, often in winter. Onset of atopic dermatitis often results in bronchial asthma or allergic rhinitis and frequently recurs even in adulthood. Serious pruritus from which atopic dermatitis patients suffer may cause reduction in adaptability to environments, vitality and work efficacy, insomnia, emotional disorders and the like, and eczematous skin lesions with accompanying pigmentation may cause skin dysmorphia and disrupt normal interpersonal relationships or social activity. In addition, dry and sensitive skin may often cause irritant contact dermatitis and restrict occupational choice.

In recent years, atopic dermatitis is increasing all over the world and increases with a sharp increase of allergic diseases in Korea. Based upon results of a survey that targeted 43,045 in total Korean elementary school students and middle school students over the country by the Korean academy of allergy and respiratory Disease in 2000, it can be seen that 24.9% of elementary school students and 12.8% of middle school students were diagnosed with atopic dermatitis. Atopic dermatitis was increased by 50% for 10 years and the increment thereof increases in proportion to age. That is, an onset rate of atopic dermatitis is high, as a level of about 17%, for adults.

Provoking causes of atopic dermatitis are known to be involved in genetic, immunologic and environmental factors. The genetic factors are atopy-related genes and immunological genes are a major part of the atopy-related genes. Atopy-related genes found to date include human leukocyte antigen (HLA) and chromosome 6p, T-cell receptor (TCR) and chromosome 7p, IgE high-affinity receptor (FcεRI-β) and chromosome 11q, and IL-4 and chromosome 5q and the like. Environmental factors include house ticks, house dust, fungi, harmful substances, such as formalin and methyl benzene, derived from materials or paints used for building of houses and exposed to the air, and chemicals contained in foods or the foods.

In addition, another aggravating factor of atopic dermatitis is Staphylococcus aureus. Clusters of Staphylococcus aureus are found in 80 to 100% of eczematous lesions of atopic dermatitis patients. This value is much higher than about 5 to about 30% which is an amount of clusters of Staphylococcus aureus found in healthy skin. Staphylococcus aureus aggravates dermatitis sites, for example, increases impetigo, infects other people and makes them susceptible to atopic dermatitis. Evidences associated with superantigenic action of exotoxin produced by Staphylococcus aureus on aggravation of atopic dermatitis are suggested. Types of exotoxin include staphylococcal enterotoxins A-D (SEA-D) and toxic shock syndrome toxin-1 (TSST-1) and these exotoxins increase production of IGE by 8 or more fold.

Examples of therapies commonly used for atopic dermatitis patients include antibiotic therapy, antihistamine therapy, steroid therapy, immunotherapy and the like. Antibiotic therapy is used for secondary infection caused by microbes such as Staphylococcus aureus described above, but has many problems associated with frequent improper administration of antibiotics and an increase in antibiotic-resistant Staphylococcus aureus. Globally important antibiotic-resistant strains include methicillin-resistant Staphylococcus aureus (MRSA), methicillin-resistant-coagulase-negative Staphylococcus aureus (MRCNS) and the like.

Accordingly, there is an increasing need for alternative substances which prevent abuse and side effects of antibiotics and generation of antibiotic-resistant bacteria, and prevent fundamental causes of atopic dermatitis and treat atopic dermatitis.

In another aspect, avian influenza is an acute viral infection adapted to birds which is at issue all over the world to date since it was firstly found in 1983 in Europe such as Belgium and France. Avian influenza causes great damage to poultry such as chickens and turkey. Animals infected with avian influenza are slaughtered in most nations and export of poultry products is restricted in the nations with infected avian influenza.

Infection is commonly caused by direct contact with guano of infected birds or via droplets, water, human feet, feed transporting vehicles, apparatuses, equipment, feces stained in egg shells and the like. Symptoms may be varied according to pathogenicity of infected virus, but common symptoms include respiratory symptoms, diarrhea and sharp decrease in spawning rate. In some cases, cyanoderma is generated in areas of the head such as the comb, facial edema is generated, feathers are concentrated at one spot and mortality rate is also varied from 0 to 100% according to pathogenicity.

In general, avian influenza virus is known to be not directly infected to humans and be infected via pig as intermediate hosts to humans, but is classified into three types of highly pathogenic avian influenza, low pathogenic avian influenza and non-pathogenic avian influenza according to pathogenicity. Of these avian influenzas, highly pathogenic avian influenza is infectious to humans as well.

A pathological mechanism of influenza virus human infection suggested associated with infection of the highly pathogenic avian influenza (HPAI) virus, H5N1, to humans, which is recently at issue demonstrates that in vivo proliferation of virus is not inhibited in an early stage due to considerable decrease in T cells and decrease in interferon-gamma upon infection of HAPI virus, viral infection metastasizes the whole body and as a result, virus is found to be proliferated in organs, respiratory organs such as the lungs as well as blood and rectum, and death of cells caused by apoptosis in these virus propagation sites, and nonpurulent pneumonia and multi-organ dysfunction caused by mass-released inflammatory cytokine are generated, thus resulting in death.

As such, damage caused by infection of avian influenza to humans becomes more serious, for example, fatality is caused by avian influenza in South-East Asia such as Vietnam and Thailand. In order to reduce this damage, much effort was made to develop avian influenza vaccines and medicines, but only some vaccines and medicines incapable of completely preventing and treating avian influenza were developed and reported. Korean Patent Publication No. 10-2007-0035203 suggests an oriental medicine composition using a Houttuynia cordata extract as an anti-avian influenza virus agent and Korean Patent No. 0485056 suggests a composition containing water-soluble glucan oligomers of yeast as a drug for indirectly preventing and treating avian influenza by facilitating production of intracellular NO and TNF-α.

Meanwhile, rice is a grain used as a main dish in Korea as well as by half or more world's human population. Production of rice in Korea is rapidly increased based on varietal improvement and development of farming techniques, but rice consumption is sharply decreased annually due to effects of western dietary culture. Such a decrease in rice consumption is due to diversification of food consumption patterns, proliferation of wellbeing culture and change of dietary habits. Another great cause of decrease in rice consumption is insufficiency of various foods using rice. That is, rice is mostly taken after primarily processed or is used in a small amount for bakery, bread and the like. Rice consumption decreases and inventory increases due to change of dietary habits caused by national income improvement and insufficient processed food.

In addition, rice contains carotene improving visual acuity, calcium and iron strengthening bones and a Monascus component inhibiting biosynthesis of cholesterol, and is well suited to Korean constitution. In particular, when rice is used in combination with milk, the milk supplements lysine, which is an amino acid lacking in rice, thus greatly improving dietary and nutritional values of fermented milk and contributing to facilitation of consumption of rice which is recently decreased in consumption.

Some foods containing rice as a main ingredient are reported to date. Korean Patent Publication No. 2002-0016109 discloses a rice beverage composition with excellent taste and a method for preparing the same wherein milled rice and brown rice are separately processed, roasting is omitted during milled rice processing, temperature, time and enzyme content upon production of starch syrup (taffy) are controlled to improve thermal stability, roasting is performed, but grinding and taffy production are omitted during brown rice processing, to prevent browning caused by heat in spite of performing retort sterilization for sufficient microbial sterilization and thereby maintain inherent white color of milled rice and minimize destruction of nutrients. In addition, Korean Patent Publication No. 2000-0006601 discloses a method of preparing a rice beverage by obtaining a clear liquid through enzyme decomposition of roasted brown rice and milled rice, wherein destruction of nutrients of brown rice and milled rice is minimized, flavors are well balanced, and the rice beverage is non-acidic, is suitable for low-temperature sterilization and is suited to fill a PET container.

However, conventional patents disclose only methods of preparing rice foods including preparing a rice saccharified liquid and simply mixing the saccharified liquid with auxiliary additives and the like, but fermented foods using lactic acid bacteria which impart beneficial components to humans were not reported and foods obtained by fermenting rice with lactic acid bacteria were developed.

Lactic acid bacteria-fermented foods using rice prevent obesity and adult diseases, facilitate propagation of intestinal microbes using indigestible carbohydrates as nutrients and deteriorate blood cholesterol, thus contributing to prevention of adult diseases. In addition, because rice contains octacosanol as a stamina (endurance) enhancer and ceramide aiding atopy improvement, rice processed foods are actively developed.

Korean Patent Publication No. 1998-075079 discloses a method of preparing a liquid rice yogurt comprising mixing 68 to 76 parts by weight of water with 5 to 18 parts by weight of an alpha rice powder, 10 to 15 parts by weight of a skimmed milk powder, 2 parts by weight of glucose and 2 parts by weight of sucrose, followed by homogenization, thermal decomposition and cooling, to obtain a sample, inoculating the sample with a mixed lactic acid bacteria seed culture, stationary-culturing the sample, and mixing the resulting fermentation raw liquid with a sugar liquid, followed by homogenization.

In addition, Korean Patent No. 0527419 discloses a rice-fermented food composition comprising a rice-fermented liquid prepared by separately adding bifidobacterium and lactic acid bacteria to a rice saccharified liquid as a main component and fermenting the resulting mixture, or adding a mixture of bifidobacterium and lactic acid bacteria and fermenting the mixture.

In addition, Korean Patent Publication No. 10-2010-0074389 discloses a rice yogurt prepared by a method including (1) a first step of preparing a rice concentrate by liquefying rice through primary enzyme treatment, saccharifying the rice through secondary enzyme treatment and concentrating the rice until a brix degree of the concentrate is 50 to 75° Bx in order to increase a content of the rice component and improve taste; (2) a second step of preparing a rice syrup by mixing the rice concentrate with sugar, dietary fibers and additives in order to mask a rice odor and improve taste; (3) a third step of preparing a lactic acid bacteria-fermented liquid by dissolving skimmed milk powder in raw milk or reconstituted milk, inoculating the solution with lyophilized mixed powdery lactic acid bacteria, fermenting the resulting solution and cooling the solution when a titrable acidity reaches 0.75%; (4) a fourth step of mixing the rice syrup of the second step with the lactic acid bacteria-fermented liquid of the third step, followed by homogenization, to balance sweetness and acidity; and (5) a fifth step of filling a plastic bottle with the mixture and packaging the plastic bottle using an automatic filling packaging machine.

However, test results demonstrating that rice-fermented food compositions obtained by fermentation of kimchi lactic acid bacteria have inhibitory activities against atopic dermatitis and influenza virus infection, in particular, inhibitory activity to propagation of avian influenza virus, other than general effects of lactic acid bacteria such as improvement of immune function, inhibition of harmful bacteria, production and maintenance of normal intestinal microflora, a decrease in blood cholesterol and anti-cancer effects, were not found. Under this circumstance, the present inventors discovered a lactic acid bacteria-fermented rice food composition prepared by inoculating a rice as a Korean main dish with Lactobacillus sakei ProBio-65 (KCTC 10755BP), which is a lactic acid bacterium extracted from kimchi, developed by the present inventors, and fermenting the resulting rice exhibits antibacterial and antiviral effects against atopic dermatitis, avian influenza virus and the like, thus completing the present invention.

### SUMMARY OF THE INVENTION

Therefore, the present invention has been made in view of the above problems, and it is an object of the present invention to provide a rice lactic acid bacteria-fermented food composition which is prepared by inoculating a rice saccharified liquid as a main component with kimchi lactic acid bacteria, followed by fermentation, and has antibacterial and antiviral effects against atopic dermatitis, avian influenza virus and the like, is free of safety problems and may be taken daily.

In accordance with an aspect of the present invention, the above and other objects can be accomplished by the provision of an antiviral composition which comprises Lactobacillus sakei ProBio-65 (accession number: KCTC 10755BP), which is a lactic acid bacteria extracted from kimchi, or a culture thereof, and exhibits an antiviral effect against avian influenza virus.

In accordance with another aspect of the present invention, there is provided a rice lactic acid bacteria-fermented food composition which comprises a rice lactic acid bacteria-fermented liquid prepared by adding Lactobacillus sakei ProBio-65 (accession number: KCTC 10755BP), which is a lactic acid bacteria extracted from kimchi, to a rice saccharified liquid, followed by fermentation and exhibits an antibacterial effect against atopic dermatitis.

In accordance with another aspect of the present invention, there is a rice lactic acid bacteria-fermented food composition which comprises a rice lactic acid bacteria-fermented liquid prepared by adding Lactobacillus sakei ProBio-65 (accession number: KCTC 10755BP), which is lactic acid bacteria extracted from kimchi, to a rice saccharified liquid, followed by fermentation, and exhibits an antiviral effect against avian influenza virus.

The present invention provides a rice lactic acid bacteria-fermented food composition prepared by inoculating a rice as a Korean main dish with Lactobacillus sakei ProBio-65 (KCTC 10755BP), followed by fermentation. The rice lactic acid bacteria-fermented food composition enables preparation of higher value-added rice fermented foods with reinforced immune function and increases availability excessively produced rice. The rice-fermented food composition exhibits remarkable effects such as antibacterial and antiviral effects against atopic dermatitis, avian influenza virus and the like, when being taken daily.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a flowchart illustrating a method for preparing a rice lactic acid bacteria-fermented food composition;

FIG. 2 is an image showing identification of antiviral activity through a cell line testing according to the present invention;

FIG. 3 is an image showing embryonated egg culturing and identification of antiviral activity according to the present invention; and

FIG. 4 is an image showing identification of antibacterial activity according to the present invention.

### DETAILED DESCRIPTION OF THE INVENTION

The present invention is characterized in providing an antiviral composition which comprises Lactobacillus sakei ProBio-65 (accession number: KCTC 10755BP), which is lactic acid bacteria extracted from kimchi, or a culture thereof, and exhibits an antiviral effect against avian influenza virus.

In addition, the present invention is characterized in providing a rice lactic acid bacteria-fermented food composition which comprises a rice lactic acid bacteria-fermented liquid prepared by adding Lactobacillus sakei ProBio-65 (accession number: KCTC 10755BP), which is a lactic acid bacteria extracted from kimchi, to a rice saccharified liquid, followed by fermentation, and exhibits an antibacterial effect against atopic dermatitis.

In addition, the present invention is characterized in providing a rice lactic acid bacteria-fermented food composition which comprises a rice lactic acid bacteria-fermented liquid prepared by adding Lactobacillus sakei ProBio-65 (accession number: KCTC 10755BP), which is lactic acid bacteria extracted from kimchi, to a rice saccharified liquid, followed by fermentation, and exhibits an antiviral effect against avian influenza virus.

Hereinafter, preferred embodiments of the present invention are provided in detail to an extent such that the embodiments may be easily executed by a person having common knowledge in the technical field to which the invention pertains. The prevent invention is implemented in various forms and is not limited to the embodiments described herein.

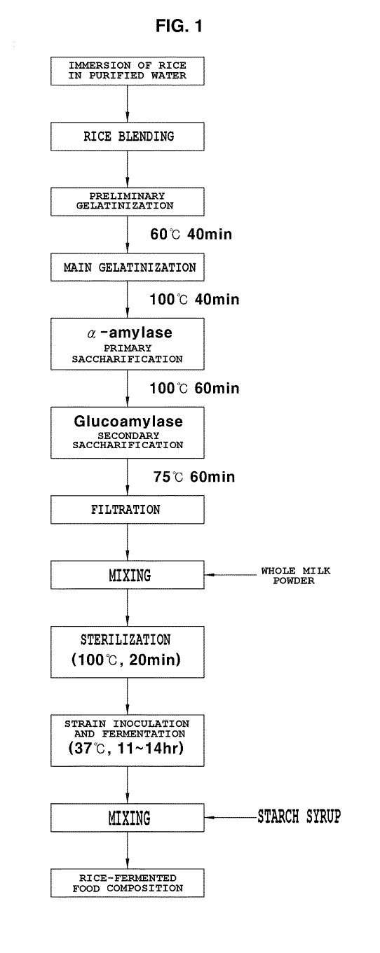

First, as shown in FIG. 1, preparation of the rice saccharified liquid may be carried out by weighing a rice, immersing the rice in purified water, blending the rice with water and gelatinizing the blend. The gelatinization may be implemented by a one-step process at about 100° C. for 20 to 40 minutes, or by a two-step process including preliminary gelatinization at about 60° C. for about 20 to 40 minutes and then main gelatinization at about 100° C. for about 20 to 40 minutes.

Next, the gelatinized liquid is primarily saccharified with α-amylase at about 80 to 100° C. for about 40 minutes to 2 hours. After the primary saccharification, the resulting liquid is secondarily saccharified with glucoamylase at about 60 to about 75° C. for about 40 minutes to 2 hours, followed by filtering to prepare a rice saccharified liquid.

Meanwhile, a kimchi liquid prepared in the home is diluted, smeared onto BCP solid media and cultured at 37° C. for 48 hours. After colonies produced after a predetermined time are observed with a microscope, 480 cocci and bacilli are selected, cultured in a MRS medium and stored at 80° C. below zero, a microbe inhibiting proliferation of Staphylococcus aureus KCTC1621 which is an aggravating factor for atopic diseases is identified, a Lactobacillus sakei ProBio-65 (KCTC 10755BP) strain is isolated and the isolated ProBio65 strain is cultured in a MRS medium at 37° C. to prepare Lactobacillus sakei ProBio-65 (KCTC 10755BP).

That is, the culturing of the ProBio-65 strain is carried out by culturing the strain in a MRS culture medium (containing glucose 2%, peptone 2%, yeast extract 0.5%, sodium acetate 0.5%, potassium phosphate dibasic 0.2%, ammonium sulfate 0.2%, magnesium sulfate 0.1%, manganese sulfate 0.005% and Tween 80 0.1%) at 37° C. for 12 hours, centrifuging the culture liquid at 13,000 rpm for 3 hours, mixing the resulting microbial cells with a hydrolyzed-skimmed milk powder at an equivalent amount, homogenizing the mixture, preliminarily freezing the mixture at −40° C., freeze-drying the mixture at an elevated temperature from −40° C. to −20° C. for 7 hours and then at −20° C. for 5 hours, at an elevated temperature from −20° C. to −5° C. for 9 hours and then at −5° C. for 10 hours, at an elevated temperature from −5° C. to 10° C. for 10 hours and then at 10° C. for 10 hours and at an elevated temperature from 10° C. to 20° C. over 8 hours, finally completing the freeze-drying at 20° C., and grinding freeze-dried microbial cells to prepare Lactobacillus sakei ProBio-65 (KCTC 10755BP).

Next, the filtered rice saccharified liquid is sterilized at 100° C. for 20 minutes, 1.5% by weight of the prepared Lactobacillus sakei ProBio-65 (KCTC 10755BP) is added to the rice saccharified liquid, fermentation is performed at 37° C. for 12 hours until a final pH reaches 4.1, and 5% by weight of a starch syrup is added thereto, to prepare a rice lactic acid bacteria-fermented food composition.

When the Lactobacillus sakei ProBio-65 is added as a fermentation starter to the prepared rice saccharified liquid, the amount of added Lactobacillus sakei ProBio-65 is preferably 0.1 to 5% by weight with respect to the weight of the rice saccharified liquid, and the rice saccharified liquid may further include at least one selected from whole milk powder, milk, skimmed soy protein and various oligosaccharides, pH adjustors and buffers, various sugars and sweeteners, and other additives and the like.

Examples of suitable oligosaccharides include isomaltooligosaccharides, galactooligosaccharides, maltooligosaccharides, cyclodextrins, oligofructoses, lactosucrose, glycosyl sucrose, gentiooligosaccharides, palatinose oligosaccharides, soy oligosaccharides, xylooligosaccharides and the like.

The pH adjusters and buffers are commonly required for liquid products and adjust pH from about 4.0 to about 6.5. Examples of the pH adjusters or buffers include weak acids such as citric acid, tartaric acid, malic acid, lactic acid and carbonic acid, and salts thereof such as sodium citrate, ammonium citrate, sodium tartrate, sodium malate, sodium lactate, calcium lactate, sodium carbonate, sodium hydrogen carbonate and the like. Sodium hydrogen phosphate may also be used as the pH adjuster or buffer. The acids and salts thereof may be used alone or in combination of two or more thereof. A mix ratio may be suitably determined while the suitable pH range is secured. Generally, the mix ratio is preferably about 2% by weight or less, more preferably 0.05 to 0.3% by weight with respect to the weight of the composition.

In addition, various sugars or sweeteners may be added to and blended with the food composition in the form of a beverage. Examples of the sugars include: monosaccharides such as glucose and fructose; disaccharides such as maltose and sucrose; polysaccharides such as dextrin and cyclodextrin; sugar alcohols such as xylitol, erythritol and sorbitol; and sugar esters. Examples of the sweeteners include natural sweeteners such as stevia extract, for example, rebaudioside A, sormatin and glycyrrhizin, and synthetic sweeteners such as saccharin and aspartame. Mix ratios of these sugars and sweeteners are commonly about 15% by weight or less, preferably about 13% by weight or less, with respect to a weight of a beverage obtained.

The food composition according to the present invention may further include one, or two or more of the following additives, if needed. Examples of the additives include juices of various fruits such as grapefruits, apples, oranges, lemons, pineapples, bananas and pears (or concentrated juices and powdery juices thereof); vitamins and provitamins (water-soluble and fat-soluble vitamins such as retinol palmitate, bisbentiamine, riboflavin, pyridoxine hydrochloride, cyanocobalamin, sodium ascorbate, nicotinic acid amide, calcium pantothenate, folic acid, biotin, cholecalciferol, choline bitartrate, tocopherol and β-carotene); flavors (such as lemon flavor, orange flavor, grapefruit flavor and vanilla essence); amino acids, nucleic acids and salts thereof (such as glutamic acid, sodium glutamate, glycine, alanine, aspartic acid, sodium aspartate and inosinic acid); plant fibers (such as polydextrose, pectin, xanthan gum, gum arabic and alginic acid); minerals and microelements (such as sodium chloride, sodium acetate, magnesium sulfate, potassium chloride, magnesium chloride, magnesium carbonate, calcium chloride, dibasic potassium phosphate, sodium phosphate, calcium glycerophospahte, sodium ferrous citrate, ferrous ammonium citrate, ferric citrate, manganese sulfate, copper sulfate, sodium iodide, potassium sorbate, zinc, manganese, copper, iodine and cobalt).

Furthermore, the additives may include grains (such as brown rice, black rice, glutinous rice, glutinous millet, sorghum, barley, mung beans, millet, Coixseed, alpha corn, maize, wheat, red beans and sesame), legumes (soybean, peanuts, yellow beans, peas and green beans), nuts (such as pine nuts, walnuts, pistachios, almonds, sunflower seeds, pecans, chestnuts and grapes), fruits (apples, citron, quince, persimmon, peach, banana, strawberry and lemon), vegetables (such as zucchini, radishes, Wuqing, cabbage, carrot, burdock, lotus root, Angelica, pine needles, paselri, myeongilyeop, dolminari and spinach), gugeunryu (such as potatoes, yacon, carrots, Codonopsis, burdock, radish, lotus root, hemp, kale and bellflower), leafy vegetables (such as Kale, Angelica and broccoli), mushrooms (such as Chaga, Ganoderma lucidum, Phellinus linteus, Hericium erinaceus, Agaricus, shiitake mushrooms, fingering mushrooms, mushroom, oyster mushroom, rock mushroom, winter mushrooms and vegetable worms), microbes (yeast, lactic acid bacteria, Bacillus bacteria and red yeast rice), microalgae (such as chlorella and spirulina), algae (such as seaweed, kelp, seaweed, laver and Chop), plant medicines (such as green tea, herbs, licorice, hwangchil, Acanthopanax, ginseng, red ginseng, Lycium, and aloe), animal medicines (such as antler and silkworms).

### EXAMPLE 1

Preparation of Rice Lactic Acid Bacteria-Fermented Food Composition

A rice was weighed, immersed in purified water, blended, preliminary gelatinized at 60° C. for 40 minutes and then gelatinized at 100° C. for 40 minutes to prepare a gelatinized liquid. The gelatinized liquid was primarily saccharified with α-amylase at 100° C. for 40 minutes. After completion of primary saccharification, the resulting liquid was secondarily saccharified with glucoamylase at 75° C. for 40 minutes to prepare a saccharified liquid, and the saccharified liquid was filtered to prepare a rice saccharified liquid.

Independent of the rice saccharified liquid, an aged kimchi liquid was diluted, smeared onto a BCP solid medium (produced by Eiken Chemical Co., Ltd., Japan), cultured at 37° C. for 48 hours, colonies produced after a predetermined time were observed with a microscope, 480 cocci and bacilli were selected, cultured in a MRS medium (produced by Difco Inc.) and stored at 80° C. below zero, a microbe inhibiting proliferation of Staphylococcus aureus KCTC1621 which is an aggravating factor of atopic diseases was identified, a Lactobacillus sakei ProBio-65 (KCTC 10755BP) strain was isolated, the isolated ProBio-65 strain was cultured in a MRS culture medium at 37° C. for 12 hours, the culture liquid was centrifuged at 13,000 rpm for 3 hours, the resulting microbial cells was mixed with a hydrolyzed skimmed milk powder, the mixture was homogenized, pre-frozen at −40° C., freeze-dried at an elevated temperature from −40° C. to −20° C. over 7 hours and then at −20° C. for 5 hours, at an elevated temperature from −20° C. to −5° C. for 9 hours and then at −5° C. for 10 hours, at an elevated temperature from −5° C. to 10° C. for 10 hours and at 10° C. for 10 hours and at an elevated temperature from 10° C. to 20° C. over 8 hours, the freeze-drying was finally completed at 20° C., and the freeze-dried microbial cells were ground to prepare Lactobacillus sakei ProBio-65 bacteria.

Whole milk powder was added to the rice saccharified liquid, in an amount of 6% by weight with respect to the weight of the rice saccharified liquid, and sterilization was performed at an elevated temperature of 100° C. for 20 minutes. 1.5% by weight of the prepared Lactobacillus sakei ProBio-65 (KCTC 10755BP) was added to the liquid, the resulting mixture was fermented at 37° C. for 12 hours until a final pH reached about 4.1, and 5% by weight of a starch syrup was added thereto to prepare a final rice lactic acid bacteria-fermented food composition.

### EXAMPLE 2

Measurement and Testing of Atopic Dermatitis SCORAD of Rice Lactic Acid Bacteria-Fermented Food Composition

The rice lactic acid bacteria-fermented food composition according to the present invention and a control food were administered to 54 mildly allergic and atopic patients over 12 weeks in human test. As a result, a severity index (SCORAD) of atopic dermatitis was analyzed and results are shown in the following Table 1.

TABLE 1
Variation in SCORAD (mean ± standard deviation)

| | Group to which rice lactic | |
|---|---|---|
| | acid bacteria-fermented | |
| | food composition is | Control food group |
| | administered (n = 27) | (n = 27) |
| SCORAD after 0 week | 39.2 ± 15.4 | 38.0 ± 17.0 |
| SCORAD after 6 weeks | 33.1 ± 17.1 | 32.5 ± 16.0 |
| SCORAD after 12 weeks | 29.6 ± 17.5 | 27.0 ± 16.8 |
| Variation in SCORAD | 9.6 ± 16.7 | 11.0 ± 15.1 |
| Improvement in | | |
| SCORAD ≧ 20% | 8 (29.6%) | 17 (70%) |

As can be seen from Table 1 above, both the group to which the rice lactic acid bacteria-fermented food composition was administered (also referred simply to “the rice lactic acid bacteria-fermented food composition group”) and the control food group were decreased in SCORAD (SCORing of Atopic Dermatitis), a clinical indicator of atopic dermatitis, but the rice lactic acid bacteria-fermented food composition group was higher in SCORAD variation than the control food group, and in particular, the rice lactic acid bacteria-fermented food composition group was administered was decreased in SCORAD by 20% or more and a ratio of patients who had improved symptoms of the rice lactic acid bacteria-fermented food composition group was administered was 70% which was two or more fold higher than 29.6% of that of the control food group.

### EXAMPLE 3

Testing of Variations in CCL17, CCL27 and CCL18 Regarding Rice Lactic Acid Bacteria-Fermented Food Composition

In addition to Example 2, CCL17 (chemokine (c-c motif) ligand 17), CCL27 (chemokine (c-c motif) ligand 27), and CCL18 (chemokine (c-c motif) ligand 18) are involved in mechanisms of mobilizing T lymphocytes in inflammation in atopic dermatitis, and concentrations of CCL17, CCL27 and CCL18 as immunological and objective indexes (biomarkers) of atopic dermatitis were measured and the results are shown in Table 2.

TABLE 2
Variations in Chemokines and Cytokines

| | Rice lactic acid bacteria- | | | | | |
|---|---|---|---|---|---|---|
| | fermented food composition group | | | Control food group | | |
| | 0 week | 12 weeks | P value | 0 week | 12 weeks | P value |
| CCL17 | 398 ± 394 | 261 ± 225 | 0.125 | 406 ± 386 | 200 ± 162 | 0.003 |
| (pg/mL) | | | | | | |
| CCL27 | 5374 ± 3289 | 4484 ± 2993 | 0.204 | 5656 ± 2538 | 3837 ± 2325 | 0.003 |
| (pg/mL) | | | | | | |
| CCL18 | 53.3 ± 15.6 | 50.8 ± 13.7 | 0.310 | 57.4 ± 21.3 | 49.9 ± 16.4 | 0.022 |
| (ng/mL) | | | | | | |

As can be seen from Table 2 above, the rice lactic acid bacteria-fermented food composition group was statistically significantly decreased in concentrations of CCL17, CCL27 and CCL18 in blood. This result clinically demonstrated that the rice lactic acid bacteria-fermented food composition according to the present invention is effective in improving symptoms of atopic dermatitis.

### EXAMPLE 4

Testing of Antiviral Activity of lactobacillus sakei Probio-65 (KCTC 10755BP)

Cell Line Culturing

MDCK (Madin-Darby canine kidney (MDCK) cell line; Korean Cell Line Bank, KCLB No. 10034) cells were cultured in Eagle's minimal essential medium (MEM) containing bovine serum and an antibiotic (100 U/ml penicillin and 100 ug/ml streptomycin) at 35° C. for 48 hours to 72 hours.

Virus Culturing

H9N2 virus (Avian influenza H9N2 virus; National Veterinary Research and Quarantine Service) was cultured in an MDCK cell line at 35° C. for 48-72 hours. MDCK monolayer cells were washed with a phosphate buffer solution twice and inoculated with the H9N2 virus, the virus was adsorbed on the cells for about 30 minutes, Eagle's minimum essential medium containing no bovine serum was added thereto, and cytopathic effects (CPE) were observed while culturing the cells at 37° C. in an incubator. When cytopathic effects were observed in about 70% of monolayer cells, infected cells containing the MEM medium were repeatedly frozen and defrosted at −70° C. three times and centrifuged, and the virus was obtained while a supernatant free of cell component was stored at −70° C.

Culturing of Lactobacillus sakei Probio-65 (KCTC 10755BP)

The culture liquid was centrifuged and a supernatant was obtained and used for testing before mixing the microbial cells with the skimmed milk powder and the freeze-drying the mixture in the process of preparing Lactobacillus sakei ProBio-65 (KCTC 10755BP) in Example 1.

Identification of Antiviral Activity

MDCK monolayer cells were seeded on a 96-well cell culture plate and washed with a phosphate buffer solution twice, the virus stored at −70° C. was diluted with by a 10-fold serial dilution method, inoculated on 10 wells in the respective dilution steps and cultured for 72 hours in a 37° C. cell incubator containing 5% CO2. At this time, cytopathic effects were observed. Based upon the results, a content of virus in the culture liquid was assayed.

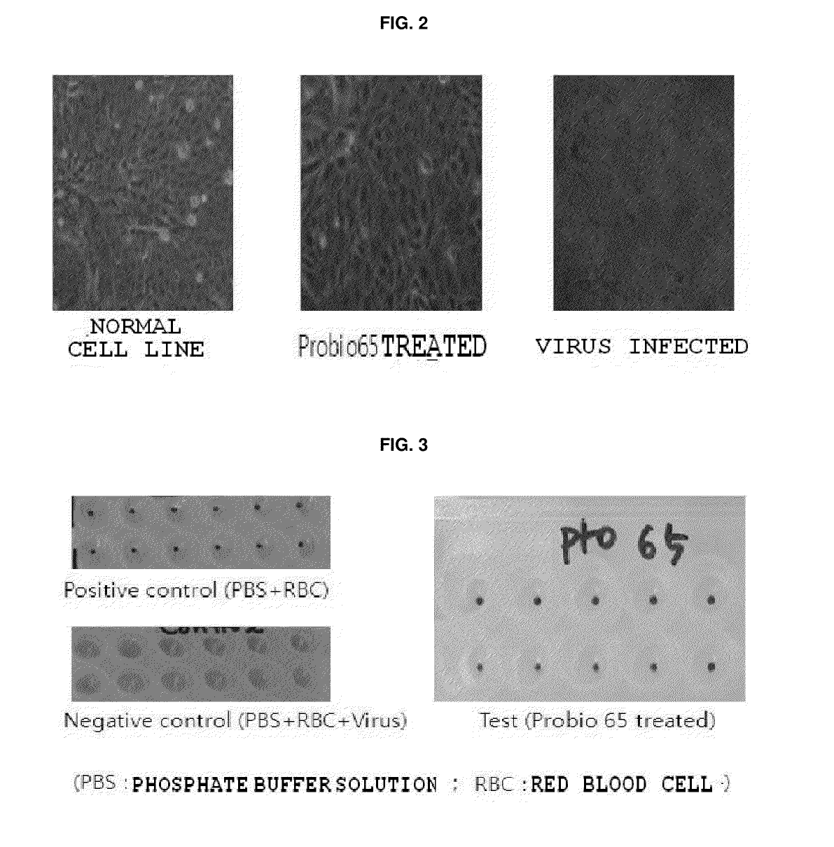

In antiviral activity testing, a virus culture was diluted with a minimum essential medium containing no bovine serum such that virus titers reached 1.0 TCID 50/0.1 mL, 10.0 TCID 50/0.1 mL, 100 TCID 50/0.1 mL and 1000 TCID 50/0.1 mL. MDCK monolayer cells were seeded on a 96-well cell culture plate and washed with a phosphate buffer solution twice. Four wells were used per Lactobacillus sakei ProBio-65 culture medium sample. 90 μl of 1.0 TCID 50/0.1 mL, 10.0 TCID 50/0.1 mL, 100 TCID 50/0.1 mL and 1000 TCID 50/0.1 mL were respectively added to first, second, third and fourth wells, and 10 μl (10%) of the Lactobacillus sakei ProBio-65 culture medium was immediately added to the each well and cultured in a 37° C. cell incubator containing 5% CO2. Cytopathic effects were observed at 24 hours, 48 hours and 72 hours after testing. In the case in which cytopathic effects are observed, the culture solution was determined to have no antiviral activity. As shown in FIG. 2, a group treated with the Lactobacillus sakei ProBio-65 culture solution exhibited no cytopathic effects, which means that the culture solution had antiviral activity.

Culturing of Embryonated Eggs and Identification of Antiviral Activity

Embryonated eggs were incubated at 37° C. in an incubator for 9 to 11 days. Inoculation sites of egg shells were determined and sites of the eggs where blood vessels were not present were inoculated with 0.2 ml of a mixture of virus and Lactobacillus sakei ProBio-65. After inoculation of the virus and lactobacillus, presence of blood vessels and shell weathering of the embryonated eggs was observed while the eggs were cultured for 4 to 5 days. An allantonic fluid was harvested from the embryonated eggs and hemagglutination reaction was performed on the allantonic fluid to identify proliferation of virus. The hemagglutination reaction was performed as follows. The respective 96-well cell culture plates were diluted with 25 μl of the allantonic fluid and 25 μl of the phosphate buffer solution by a 2-fold serial dilution method. 25 μl of 1% red blood cells isolated from chicken were seeded on the diluted 96-well cell culture plates and hemagglutination reaction was observed at room temperature for 40 minutes. In the case in which, during embryonated egg testing, hemagglutination was observed and blood cells were deposited on the 96-well cell culture plates, the culture solution was determined to have no antiviral activity. As shown in FIG. 3, a group treated with the Lactobacillus sakei ProBio-65 culture medium was determined to have no antiviral activity because hemagglutinated substances were not deposited on the 96-well cell culture plates.

### EXAMPLE 5

Testing of Antiviral Activity of Rice Lactic Acid Bacteria-Fermented Food Composition

The rice lactic acid bacteria-fermented food composition prepared in Example 1 was tested in the same manner as the testing of antiviral activity of Lactobacillus sakei ProBio-65 (accession number: KCTC 10755BP) in Example 4. As a result, the same result as in Example 4 was obtained.

### EXAMPLE 6

Testing of Antibacterial Activity of Rice Lactic Acid Bacteria-Fermented Food Composition

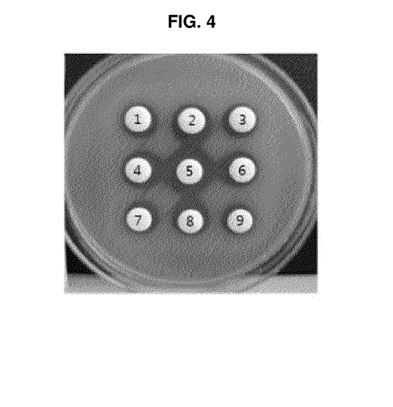

The rice lactic acid bacteria-fermented food composition prepared in Example 1 was tested in the same manner as the testing of antibacterial activity of Lactobacillus sakei ProBio-65 (accession number: KCTC 10755BP) in Example 3. The obtained result is shown in Table 2. As can be seen from Table 3 and FIG. 4, the antibacterial activity was confirmed.

TABLE 3

| | | Staphylococcus aureus |
|---|---|---|
| | | HSAc |
| | | Paper disc |
| No. | Strain | (8 mm diameter) |
| 1 | Lactobacillus ruteri, isolate | 11 |
| 2 | Lactobacillus plantarum, isolate | 14 |
| 3 | Lactobacillus acidophilus; isolate | 13 |
| 4 | Lactobacillus bunchneri, isolate | 15 |
| 5 | Lactobacillus sakei, isolate | 17 |
| 6 | Lactobacillus rhamnosus KCTC 5033* | 15 |
| 7 | Lactobacillus sakei subsp. sakei KCTC 3603^{T} | 14 |
| 8 | Lactobacillus paracasei subsp. paracasei | 15 |
| | KCTC 3510^{T} | |
| 9 | Lactobacillus brevis KCTC 3102 | 12 |

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

The present invention provides a rice lactic acid bacteria fermented food composition which is prepared by adding kimchi lactobacillus to a rice saccharified liquid as a main ingredient and fermenting the mixture, and has antibacterial and antiviral effects against atopic dermatitis and avian influenza virus, and a functional health food comprising the same, thus having industrial applicability.

1. A rice lactic acid bacteria-fermented food composition comprising a rice lactic acid bacteria-fermented liquid prepared by adding Lactobacillus sakei ProBio-65 (accession number: KCTC 10755BP), as lactic acid bacteria extracted from kimchi, to a rice saccharified liquid, followed by fermentation, the rice lactic acid bacteria-fermented food composition having an antibacterial effect against atopic dermatitis,
wherein the rice saccharified liquid is prepared by weighing a rice, immersing the rice in purified water, blending the rice and gelatinizing the blend at 100° C. for 20 to 40 minutes or preliminarily gelatinizing the blend at 60° C. for 20 to 40 minutes and then main-gelatinizing the blend at 100° C. for 20 to 40 minutes to prepare a gelatinized liquid, primarily saccharifying the gelatinized liquid with α-amylase at 80 to 100° C. for 40 minutes to 2 hours and secondarily saccharifying the resulting liquid with glucoamylase at 60 to 75° C. for 40 minutes to 2 hours, followed by filtering.

2. A rice lactic acid bacteria-fermented food composition comprising a rice lactobacillus fermented liquid prepared by adding Lactobacillus sakei ProBio-65 (accession number: KCTC 10755BP), as lactic acid bacteria extracted from kimchi, to a rice saccharified liquid, followed by fermentation, the rice lactic acid bacteria-fermented food composition having an antiviral effect against avian influenza virus,
wherein the rice saccharified liquid is prepared by weighing a rice, immersing the rice in purified water, blending the rice and gelatinizing the blend at 100° C. for 20 to 40 minutes or preliminarily gelatinizing the blend at 60° C. for 20 to 40 minutes and then main-gelatinizing the blend at 100° C. for 20 to 40 minutes to prepare a gelatinized liquid, primarily saccharifying the gelatinized liquid with α-amylase at 80 to 100° C. for 40 minutes to 2 hours and secondarily saccharifying the resulting liquid with glucoamylase at 60 to 75° C. for 40 minutes to 2 hours, followed by filtering.

3. A method for preparing the rice lactic acid bacteria-fermented food composition according to claim 1 comprising:
gelatinization comprising weighing a rice, immersing the rice in purified water, blending the rice and gelatinizing the blend at 100° C. for 20 to 40 minutes or preliminarily gelatinizing the blend at 60° C. for 20 to 40 minutes and then main-gelatinizing the blend at 100° C. for 20 to 40 minutes to prepare a gelatinized liquid;
rice saccharification comprising primarily saccharifying the gelatinized liquid with α-amylase at 80 to 100° C. for 40 minutes to 2 hours and secondarily saccharifying the resulting liquid with glucoamylase at 60 to 75° C. for 40 minutes to 2 hours, followed by filtering to prepare a rice saccharified liquid;
mixing and sterilization comprising adding at least one additive selected from whole milk powder, milk, skimmed soy protein, oligosaccharides, pH adjusters, buffers, sugars, sweetenings, fruit juices, vitamins, sweeteners, amino acids, nucleic acids and salts thereof, plant fibers and minerals to the rice saccharified liquid, followed by sterilization at 100° C. for 20 minutes;
preparing Lactobacillus sakei ProBio-65 (accession number: KCTC 10755BP) as lactic acid bacteria, comprising diluting an aged kimchi liquid, smearing the kimchi diluted solution onto a BCP solid medium, culturing the kimchi diluted solution at 37° C. for 48 hours, culturing selected cocci and bacilli in a MRS medium, storing the cocci and bacilli at 80° C. below zero, isolating a Lactobacillus sakei ProBio-65 (KCTC 10755BP) strain and culturing the isolated ProBio-65 strain in the MRS medium at 37° C. to prepare Lactobacillus sakei ProBio-65 bacteria; and
fermenting the lactic acid bacteria comprising adding the Lactobacillus sakei ProBio-65 (accession number: KCTC 10755BP) as the lactic acid bacteria to the mixed sterilized rice saccharified liquid prepared in the mixing and sterilization, in an amount of 0.1 to 5% by weight with respect to the weight of the rice saccharified liquid, followed by fermenting at 37° C. for 12 hours until a final pH reaches 4.1.

4. The method according to claim 3, wherein in the step of the preparation of the Lactobacillus sakei ProBio-65 (accession number: KCTC 10755BP) as the lactic acid bacteria, the culturing of the Lactobacillus sakei ProBio-65 (accession number: KCTC 10755BP) strain and the preparation of the lactic acid bacteria are carried out by culturing the Lactobacillus sakei ProBio-65 strain in the MRS medium (containing glucose 2%, peptone 2%, yeast extract 0.5%, sodium acetate 0.5%, potassium phosphate dibasic 0.2%, ammonium sulfate 0.2%, magnesium sulfate 0.1%, manganese sulfate 0.005% and Tween 80 0.1%) at 37° C. for 12 hours, centrifuging the culture liquid at 13,000 rpm for 3 hours, mixing the resulting microbial cells with a hydrolyzed-skimmed milk powder at an equivalent amount, homogenizing the mixture, preliminarily freezing the mixture at −40° C., freeze-drying the mixture at an elevated temperature from −40° C. to −20° C. for 7 hours and then at −20° C. for 5 hours, at an elevated temperature from −20° C. to −5° C. for 9 hours and then at −5° C. for 10 hours, at an elevated temperature from −5° C. to 10° C. for 10 hours and then at 10° C. for 10 hours and at an elevated temperature from 10° C. to 20° C. over 8 hours, completing the freeze-drying at 20° C., and grinding freeze-dried microbial cells to prepare the Lactobacillus sakei ProBio-65 (KCTC 10755BP).

5. A method for preparing the rice lactic acid bacteria-fermented food composition according to claim 2 comprising:
gelatinization comprising weighing a rice, immersing the rice in purified water, blending the rice and gelatinizing the blend at 100° C. for 20 to 40 minutes or preliminarily gelatinizing the blend at 60° C. for 20 to 40 minutes and then main-gelatinizing the blend at 100° C. for 20 to 40 minutes to prepare a gelatinized liquid;
rice saccharification comprising primarily saccharifying the gelatinized liquid with α-amylase at 80 to 100° C. for 40 minutes to 2 hours and secondarily saccharifying the resulting liquid with glucoamylase at 60 to 75° C. for 40 minutes to 2 hours, followed by filtering to prepare a rice saccharified liquid;
mixing and sterilization comprising adding at least one additive selected from whole milk powder, milk, skimmed soy protein, oligosaccharides, pH adjusters, buffers, sugars, sweetenings, fruit juices, vitamins, sweeteners, amino acids, nucleic acids and salts thereof, plant fibers and minerals to the rice saccharified liquid, followed by sterilization at 100° C. for 20 minutes;
preparing Lactobacillus sakei ProBio-65 (accession number: KCTC 10755BP) as lactic acid bacteria, comprising diluting an aged kimchi liquid, smearing the kimchi diluted solution onto a BCP solid medium, culturing the kimchi diluted solution at 37° C. for 48 hours, culturing selected cocci and bacilli in a MRS medium, storing the cocci and bacilli at 80° C. below zero, isolating a Lactobacillus sakei ProBio-65 (KCTC 10755BP) strain and culturing the isolated ProBio-65 strain in the MRS medium at 37° C. to prepare Lactobacillus sakei ProBio-65 bacteria; and
fermenting the lactic acid bacteria comprising adding the Lactobacillus sakei ProBio-65 (accession number: KCTC 10755BP) as the lactic acid bacteria to the mixed sterilized rice saccharified liquid prepared in the mixing and sterilization, in an amount of 0.1 to 5% by weight with respect to the weight of the rice saccharified liquid, followed by fermenting at 37° C. for 12 hours until a final pH reaches 4.1.

6. The method according to claim 5, wherein in the step of the preparation of the Lactobacillus sakei ProBio-65 (accession number: KCTC 10755BP) as the lactic acid bacteria, the culturing of the Lactobacillus sakei ProBio-65 (accession number: KCTC 10755BP) strain and the preparation of the lactic acid bacteria are carried out by culturing the Lactobacillus sakei ProBio-65 strain in the MRS medium (containing glucose 2%, peptone 2%, yeast extract 0.5%, sodium acetate 0.5%, potassium phosphate dibasic 0.2%, ammonium sulfate 0.2%, magnesium sulfate 0.1%, manganese sulfate 0.005% and Tween 80 0.1%) at 37° C. for 12 hours, centrifuging the culture liquid at 13,000 rpm for 3 hours, mixing the resulting microbial cells with a hydrolyzed-skimmed milk powder at an equivalent amount, homogenizing the mixture, preliminarily freezing the mixture at −40° C., freeze-drying the mixture at an elevated temperature from −40° C. to −20° C. for 7 hours and then at −20° C. for 5 hours, at an elevated temperature from −20° C. to −5° C. for 9 hours and then at −5° C. for 10 hours, at an elevated temperature from −5° C. to 10° C. for 10 hours and then at 10° C. for 10 hours and at an elevated temperature from 10° C. to 20° C. over 8 hours, completing the freeze-drying at 20° C., and grinding freeze-dried microbial cells to prepare the Lactobacillus sakei ProBio-65 (KCTC 10755BP).
